(19) United States
(12) Patent Application Publication
(10) Pub. No.: US 2013/0085555 A1
(43) Pub. Date: Apr. 4, 2013

(54) SYSTEM AND METHOD FOR TARGETED LIPOLYSIS

(71) Applicant: Savvier, LP, Carlsbad, CA (US)

(72) Inventor: Jeff Tuller, San Marcos, CA (US)

(73) Assignee: Savvier, LP, Carlsbad, CA (US)

(21) Appl. No.: 13/632,481

(22) Filed: Oct. 1, 2012

Related U.S. Application Data
(60) Provisional application No. 61/541,982, filed on Sep. 30, 2011.

(51) Int. Cl. A61F 7/08

(52) U.S. Cl. CPC A61F 7/08
USPC 607/112

(57) ABSTRACT

A system and method for targeted lipolysis on a portion of a human body is provided comprising a resilient, expandable, and flat wrap oriented around the targeted portion of a human body; the wrap is configured to increase the external temperature of the targeted portion of the human body; and the wrap further is configured to provide compression to the targeted portion of the human body by stretching a material that is included in a portion of the wrap.

The invention relates generally to the field of system and method for inducing lipolysis at targeted body areas for humans.

### BACKGROUND OF THE INVENTION

It is well known that current society obsesses over physical appearance with magazines and media portraying the optimal body having a slim midsection, i.e., an attractive belly. However, overweight and obesity trends are heading in the opposite direction with obesity increasing every decade for the past 40 years. Common practice is to utilize diets and exercise for reducing weight, reducing body fat, and slenderizing the belly. It is well known that diet and exercise are useful for reduction of excess body fat.

However, diets and exercise cannot solve all issues related to excess body fat. Highly restrictive diets are difficult to follow, particularly when the dieter does not have a support system in place. More importantly, diets tend to affect all parts of the body equally. Depending on a person's genetic heritage and history, body fat may tend to accumulate in particular areas even in persons whose weight is within the range considered as normal. It is well known that many people may find it difficult to reduce excess fat in specific areas. For this reason, targeted fat reduction apparatus and techniques have been created.

Targeted fat reduction techniques include both targeted exercise; electrical muscle stimulation (particularly in devices such as belts); and surgical procedures, such as liposuction. While exercise can focus on specific body parts to provide muscle strengthening and toning, exercise acts similarly to diet with regard to fat removal, and it tends to reduce fat systemically (all across the body and less so from a targeted perspective); thus, exercise tends not to be as effective in targeting fat reduction as liposuction. However, not everyone is a good candidate for liposuction. Further, liposuction can have many harmful side effects, such as infection and even mortality. Even though exercise belts are effective, exercise belts, particularly those that incorporate electrical stimulation, are not suitable to be worn for long periods of time due to their discomfort and bulkiness. Thus, it would be highly desirable to have apparatus and methods for targeted lipolyis that is suitable for a wider variety of subjects, and which is faster and more comfortable than electrical stimulation belts, and does not have the cost or side effects of liposuction.

### SUMMARY OF THE INVENTION

The system and method for targeted lipolysis on a portion of a human body solves the above problems as well as other problems of the prior art.

The system for targeting lipolysis on a portion of a human body comprises a resilient, expandable, flat wrap, configured to induce lipolysis when oriented around the portion of a human body, the wrap configured to provide compression to the portion of the human body by stretching a material that is included in a portion of the wrap; the wrap further is configured to increase the external temperature of the targeted portion of the human body. Preferably, the material comprises a plurality of woven sections. More preferably, at least one of the woven sections is a substantially oval section. More preferably, at least one of the woven sections is a substantially round section. More preferably, at least one of the woven sections is a substantially rectangular section. Preferably, the material is configured to be breathable. Preferably, the material is configured to occlusively seal the targeted portion of the human body. Preferably, the system for targeting lipolysis encases the abdominal region of the human body. Preferably, the system for targeting lipolysis encases the buttocks region of the human body. Preferably, the system for targeting lipolysis encases a thigh region of the human body. Preferably, the material of the wrap is more resilient at, near, or along at least some of its sides compared to at or near its center. Preferably, the system for targeted lipolysis comprises lipolytic cream. More preferably, the wrap is configured to release lipolytic agents from said lipolytic cream. Preferably, the system for targeted lipolysis is configured to release lipolytic agents from said lipolytic cream. More preferably, the lipolytic agent is selected from the group consisting of caffeine, aminophylline, coco extracts, kola nut extracts, and xanthines, or combinations thereof.

A method for targeting lipolysis on a portion of a human body also is provided, said method comprising applying a lipolytic cream to said portion of a human body; orienting a resilient, expandable, flat wrap over said portion of a human body; and increasing the temperature of said portion of a human body. Preferably, the temperature increases from 33° C. to 35° C. Preferably, the wrap is configured to release lipolytic agents from the lipolytic cream. Preferably, the human performs muscle exercises on said targeted portion of a human body. More preferably, the human performs muscle exercises for at least two minutes. Preferably, the wrap is oriented over said portion of a human body for about 5 minutes to 12 hours. Preferably, the method further comprises occlusively sealing the targeted portion of a human body.

It has been found that the combination of the wrap, the lipolytic cream, and exercise is particularly effective in removing targeted excess fat. It is also more comfortable than electrical stimulation (particularly in devices such as belts), and not as potentially dangerous as liposuction. Numerous other advantages and features of the invention will become apparent from the following detailed description.

### BRIEF DESCRIPTION OF THE DRAWINGS

FIG. 1 illustrates a picture of a wrap for targeting lipolysis positioned on a human body.

FIG. 2 illustrates a picture of a back view of the wrap for targeting lipolysis.

FIG. 3 illustrates a picture of a front view of the wrap for targeting lipolysis.

FIG. 4 is a flow chart illustrating a method according to a preferred embodiment of the invention.

FIG. 5 is a line graph illustrating the temperature increase on the skin of the wearer of the wrap for targeting lipolysis.

### DETAILED DESCRIPTION OF THE PREFERRED EMBODIMENT

In this disclosure, the term “lipolysis” means the breakdown of lipids and involves the hydrolysis of triglycerides into free fatty acids followed by further degradation into acetyl units by beta oxidation. Lipolysis dissolves small to medium unwanted localized areas of fat.

In the preferred embodiment, the system for targeted lipolysis comprises a wrap. Although a wrap is preferred to target lipolysis, other similar types of expandable items may be used including, but not limited to, bands and belts. Preferably, the wrap is configured to target lipolysis when oriented around a portion of a human body. The wrap to target lipolysis may be configured to operate on multiple portions of the human body including, but not limited to, the abdominal region, the buttocks region, the thigh region, and/or all of the above such as a full or mid-body wrap. The wearer orients the wrap by pulling and stretching the wrap. It should be noted that the wrap will recover its original elasticity and shape even after it is pulled and stretched. The wrap is configured to provide compression to the portion of the human body by stretching a material that is included in a portion of the wrap. As described below, the compression also provides occlusion thereby enhancing the absorption of lipolytic agents.

In an embodiment, the wrap is resilient in at least its lengthwise direction. The wrap is also resilient in at least its widthwise direction. “Resilient” as used herein is the ability of the material to absorb energy when it is deformed elastically, and release that energy upon unloading.

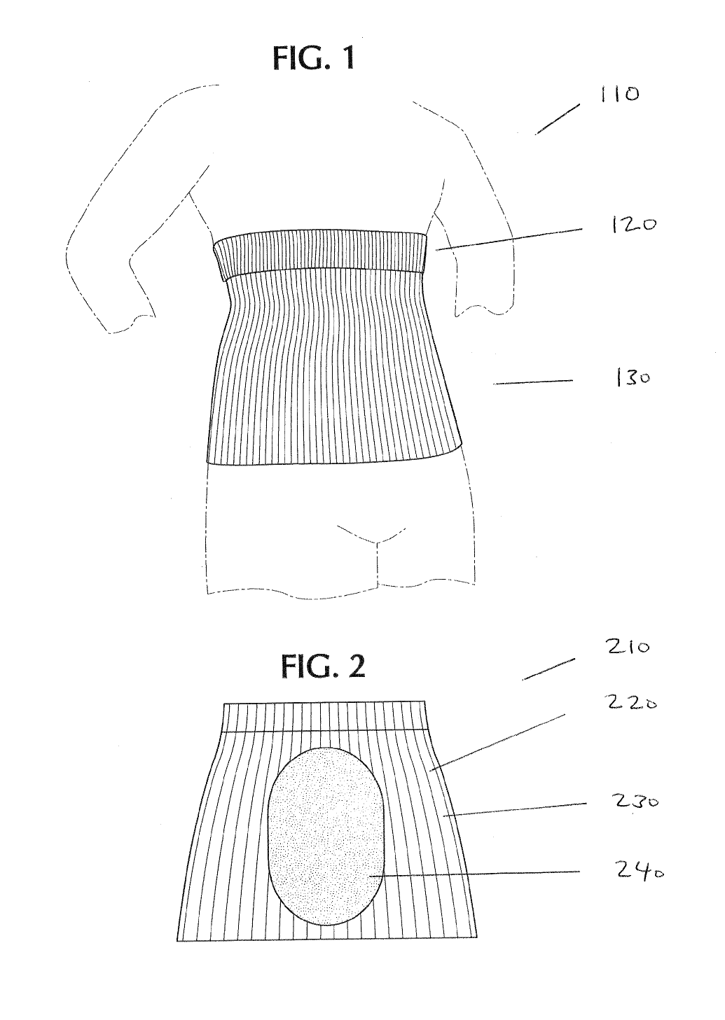

With reference to FIG. 1, an exemplary wrap 110 fully encases the belly region of the wearer. The wrap is comprised of wide top section 120 that holds the wrap in place and a relatively elongated bottom section 130. The wrap expands and contracts according to the contours of the human wearing the wrap. Preferably, the wide top band is more flexible relative to the bottom section. During expansion, the wrap extends at least laterally outwardly and at least vertically from the portion of the human body on which the wrap is placed. The expansion is determined by the elasticity of the woven materials described below. When the wrap is oriented on the portion of the human body, the wrap exerts an inward compression on that portion of the human body.

The wrap to target lipolysis may comprise a plurality of woven sections. Preferably, this is achieved by using materials comprising, but not limited to, fabric, polymers, composites, nylon, and spandex. Any one or a combination of these materials may be woven together to manufacture the wrap. The types of weave include rib, matt, twill, plain, and basket.

The combination of the woven materials and compression occlusively seal the portion of the human body on which the wrap is placed. The term “occlusively” as used herein refers to a feature of the woven materials to provide an air and water seal. Such seal may be partial, such as a semi-breathable weave, or a full seal (i.e., semi-occlusion to full occlusion). “Breathable” as used herein means the ability of the material to allow air, water, or both to pass through it.

With reference to FIG. 2, in an embodiment, a section of the wrap in contact with the posterior portion of the wearer comprises a mixed weave, that is, a ribbed weave 220, then a plain weave 230 in a repeating pattern. In an embodiment, the wrap may be comprised of at least one substantially oval section 240 on the side that is positioned at the back of the body during wear. The oval section 240 may be comprised of a weave that is different from the weave of other sections of the wrap. The sections may vary in compression relative to the other sections. The novel oval shape allows the wearer flexibility of movement in all directions during exercise, walking, sitting, or lying down. This section also improves the wearer's posture and provides an erect figure for better appearance. In another embodiment, the wrap comprises at least one substantially circular section. In yet another embodiment, the wrap comprises at least one substantially rectangular section.

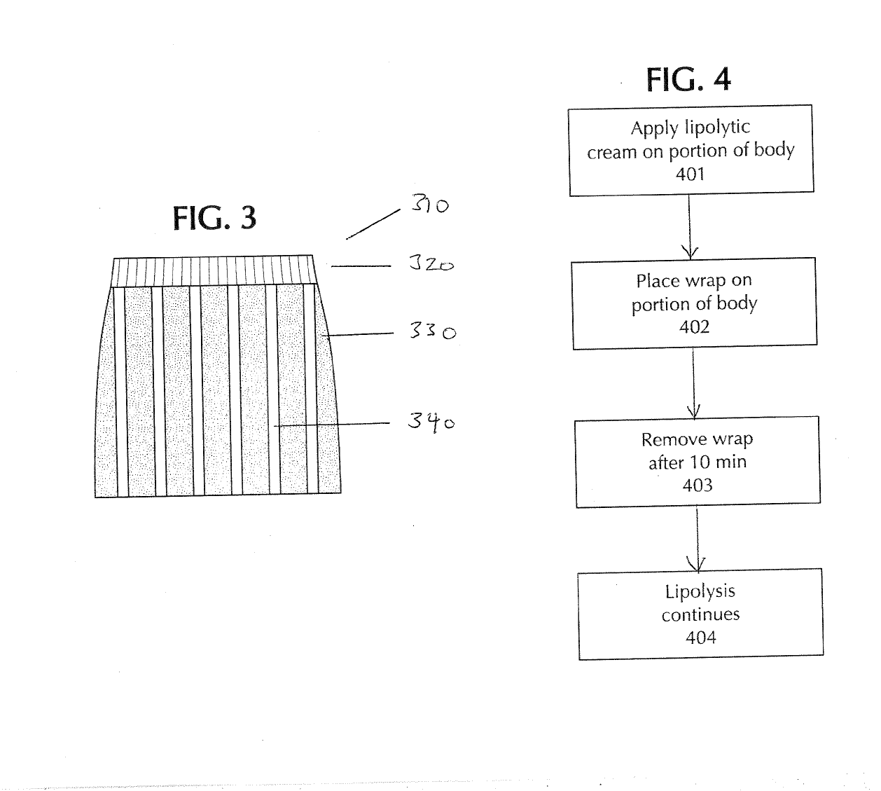

In another embodiment, as shown in FIG. 3, the wrap in contact with a portion of the wearer 310 comprises a top section 320, and a bottom section comprising a mixed weave, that is, a ribbed weave 340, then a twill weave 230 in a repeating pattern. The rib weave of the top section 320 may be less elastic in the outward direction relative to the bottom section.

In a preferred embodiment, the wrap for targeting lipolysis on a portion of a human body is used in combination with a lipolytic agent that is applied to the portion of the wearer prior to orientation of the wrap. The lipolytic agent includes, but is not limited to, caffeine, aminophylline, coco extracts, kola nut extracts, and xanthines, or combinations thereof.

With reference to the flow diagram of FIG. 4, a wearer of the wrap applies a lipolytic cream comprising a lipolytic agent on a portion of the body at step 401. The wrap then is placed on the portion of the body at step 402. Although not shown, in an embodiment, the wearer conducts muscle exercises for about two minutes or longer. These exercises include, but are not limited to, isometric contractions, standing abdominal contractions, curls, and sit-ups, where the muscles being flexed are within the wrapped area. After about ten minutes, the wrap is removed at step 403. In other embodiments, the wrap also may be oriented on the portion of the human body from about 5 minutes to 12 hours. After the wrap is taken off, the lipolytic agent has absorbed into the dermal layers penetrating the subcutaneous fat layers, and lipolysis continues at step 404.

The lipolytic agent to be applied may be encapsulated within liposomes. Liposomes are the most widely used carrier systems in medicine. The targeting of the carrier is not effective if the encapsulated lipolytic agent within the liposome is not released at the intended site or at the appropriate time. The compression and woven features of the system for targeting lipolysis described herein combine to release the lipolytic agent from the liposome at the intended site and at the appropriate time. For example, caffeine pathways have been shown to predominantly absorb via hair follicles. The occlusion and compression of the wrap stretches and flexes the wrap across the skin as the user wears the wrap, thus sliding cream into contact with hair follicles during the treatment period to enhance absorption.

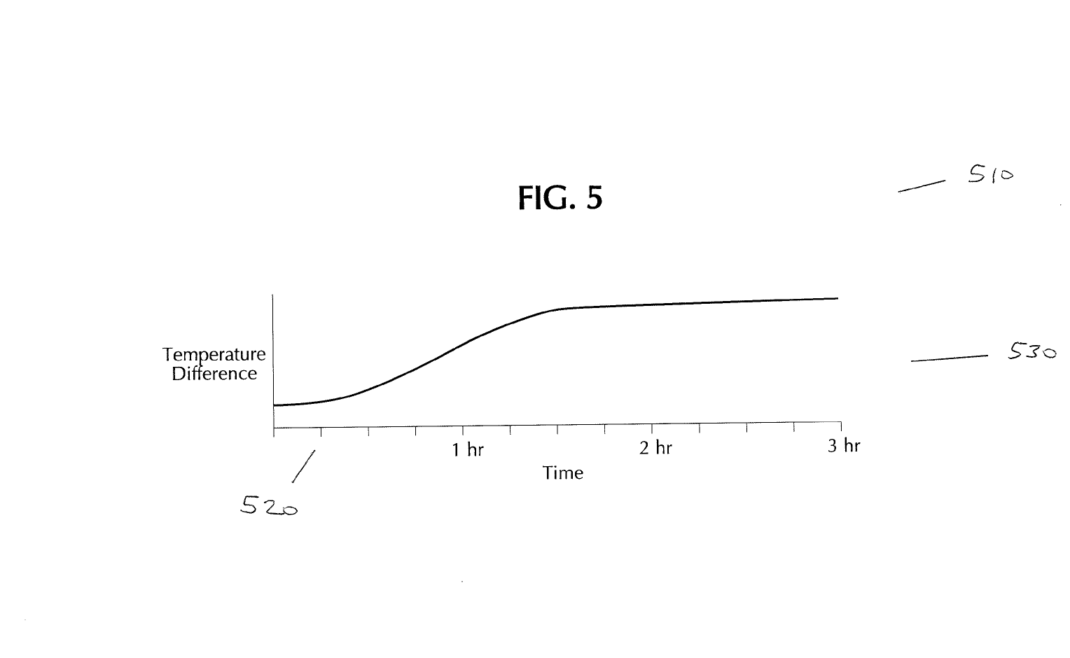

The efficacy of the system and method for targeting lipolysis on the abdominal region of a human body has been confirmed in a joint clinical study with two medical universities. Using heat vision photography, temperature measurements of the skin of the belly were taken. First, lipolytic cream was applied on the belly and the wrap was oriented. The wrap was taken off after ten minutes and the temperature again was measured. As shown in FIG. 5, the time-lapse sequence 510 shows the temperature initially rises and then stabilizes for over three hours. The temperature gradually rises for the first ten minutes (520) from about 33° C. to about 35° C. At or around this point, the lipolytic agents are released from the lipolytic cream and the temperature stabilized to only a few degrees (530) above the initial temperature so it is comfortable and unnoticeable to most people. The wrap was taken off (520). During the entire test, the temperature range of the skin of the wearer's belly region was measured to increase from 33° C. to 37° C. (shown at 530).

To measure the lipolytic activity, ultrasound imaging was used to measure the thickness of the subcutaneous fat in the abdominal region of the human subject over the course of one month. Results of subcutaneous fat loss by wearers of the wrap without undergoing any exercise are summarized in Table 1.

TABLE 1
Subcutaneous fat loss by wearers of the wrap without exercise

| | Loss of subcutaneous fat in the |
|---|---|
| Time | abdominal region (without exercise) |
| 1 week | 0.35 inches |
| 30 days | 1 inch |

The studies also found that subcutaneous fat loss by wearers of the wrap with exercise/diet is approximately twice the fat loss compared to subjects following the same exercise/diet alone without wearing the wrap.

There has been described a system and method for targeting lipolysis on a portion of a human body which have been proven to be more effective than the prior art and having numerous novel features. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by the targeted lypolysis system and methods described.

It should also be noted that ratios, concentrations, amounts, and other numerical data may be expressed herein in a range format. It is to be understood that such a range format is used for convenience and brevity and, thus, should be interpreted in a flexible manner to include not only the numerical values explicitly recited as the limits of the range, but also to include all of the individual numerical values or sub-ranges encompassed within that range as if each numerical value and sub-range is explicitly recited.

I claim:

1. A system for targeted lipolysis on a portion of a human body, comprising
a resilient, expandable, and flat wrap oriented around said targeted portion of a human body;
wherein said wrap is configured to increase the external temperature of the targeted portion of the human body; and
said wrap is further configured to provide compression to said targeted portion of the human body by stretching a material that is included in a portion of the wrap.

2. A system for targeted lipolysis on a portion of a human body as in claim 1 wherein said material comprises a plurality of woven sections.

3. A system for targeting lipolysis on a portion of a human body as in claim 1 wherein said material is configured to be breathable.

4. A system for targeting lipolysis on a portion of a human body as in claim 1 wherein said material is configured to occlusively seal the targeted portion of the human body.

5. A system for targeted lipolysis on a portion of a human body as in claim 2 wherein at least one of said woven sections is a substantially oval section.

6. A system for targeted lipolysis on a portion of a human body as in claim 2 wherein at least one of said woven sections is a substantially round section.

7. A system for targeted lipolysis on a portion of a human body as in claim 2 wherein at least one of said woven sections is a substantially rectangular section.

8. A system for targeted lipolysis on a portion of a human body as in claim 1 wherein said wrap encases the abdominal region of the human body.

9. A system for targeted lipolysis on a portion of a human body as in claim 1 wherein said wrap encases the thigh region of the human body.

10. A system for targeted lipolysis on a portion of a human body as in claim 2 wherein said wrap is more resilient at, near, or along at least some of its sections.

11. A system for targeted lipolysis on a portion of a human body as in claim 1 wherein said system comprises a lipolytic cream.

12. A system for targeted lipolysis on a portion of a human body as in claim 11 wherein said wrap is configured to release lipolytic agents from said lipolytic cream.

13. A system for targeted lipolysis on a portion of a human body as in claim 12 wherein said lipolytic agent is selected from the group consisting of caffeine, aminophylline, coco extracts, kola nut extracts, and xanthines, or combinations thereof.

14. A method for targeted lipolysis on a portion of a human body, said method comprising:
applying a lipolytic cream to said targeted portion of a human body;
orienting a resilient, expandable, flat wrap over said targeted portion of a human body; and
increasing the external temperature of said targeted portion of a human body.

15. A method as in claim 14 wherein said temperature increases from 33° C. to 35° C.

16. A method as in claim 14 wherein said method further comprises releasing lipolytic agents from said lipolytic cream.

17. A method as in claim 14 wherein said method further comprises performing muscle exercises on said targeted portion of a human body.

18. A method as in claim 17 wherein said performing muscle exercises on said targeted portion of a human body is for at least two minutes.

19. A method as in claim 14 wherein said wrap is oriented over said portion of a human body for about 5 minutes to 12 hours.

20. A method as in claim 14, said method further comprising occlusively sealing said targeted portion of a human body.
